(19) United States
(12) Patent Application Publication
(10) Pub. No.: US 2023/0197189 A1
(43) Pub. Date: Jun. 22, 2023

(54) COMPUTATIONAL TECHNIQUES FOR IDENTIFYING UNOBSERVED HEREDITARY INFORMATION BASED ON ANALYSIS OF LIMITED DATA

(71) Applicant: ABS Global, Inc., DeForest, WI (US)

(72) Inventors: Benjamin Brett, Sun Prairie, WI (US); Matthew Campbell, Madison, WI (US); Patrice Linel, DeForest, WI (US); Steve Rounsley, Waunakee, WI (US)

(73) Assignee: ABS Global, Inc., DeForest, WI (US)

(21) Appl. No.: 18/066,940

(22) Filed: Dec. 15, 2022

Related U.S. Application Data
(60) Provisional application No. 63/291,112, filed on Dec. 17, 2021.

(51) Int. Cl. G16B 20/00; G16B 30/00

(52) U.S. Cl. CPC G16B 20/00; G16B 30/00

(57) ABSTRACT

Systems and methods for organism genotyping and using genomic data for genotype imputation are disclosed. The system can maintain first genetic sequence information of a sire and second genetic sequence information for a dam, the first and second genetic sequence information indicating one or more single nucleotide polymorphisms (SNPs) of interest. The system can sequence, based on a skim sequencing technique, a sample of genetic information of a progeny of the sire and the dam. The system can identify informative variants based on the first genetic sequence information and the second genetic sequence information. The system can identify informative reads in the sequence of the progeny based on the informative variants. The system can construct a genotype of the progeny based on the informative reads and the one or more SNPs of interest in the first and second genetic sequence information.

### CROSS REFERENCE TO RELATED APPLICATION

This application claims the benefit of and priority to U.S. Provisional Application 63/292,112 filed on Dec. 17, 2021. 63/291,112 is hereby incorporated by reference in its entirety.

### TECHNICAL FIELD

The present technology relates to systems and methods for organism genotyping and using genomic data for genotype imputation.

### BACKGROUND

Genome sequencing can provide useful insights into the genetic properties of living organisms. Genome sequencing of embryos is challenging, because the large amount of genetic material required to successfully sequence the genome of the embryo would compromise the viability of the organism. In addition, the cost associated with sequencing the entire genome for every embryo would be expensive.

Another challenge in genome sequencing is that the process is typically time consuming. When performing genome sequencing, gene analysts are in a race against embryonic development. For example, biopsies of in vivo bovine embryos cannot be completed until day 4-5 of embryonic development, the morulae stage, because this is when the embryo enters the uterus and is accessible for collection by non-surgical means. Whereas in vitro, although the embryo is readily available for genomic analysis prior to day 4-5 of embryonic development, sample collection before this timeframe would significantly decrease the chance of embryo survival post-biopsy. Therefore, both in vivo and in vitro sample collection for genome sequencing begins at the earliest at day 4-5 of embryonic development. This sample collection timeframe is crucial because at the blastocyst hatching stage of embryonic development, which typically occurs at day 7, the embryo leaves the zona pellucida and the survival of these embryos after freezing is diminished. In addition, embryos at the blastocyst hatching stage experience reduced pregnancy potential when vitrified. Therefore, storage techniques for later embryo use are not recommended for embryos at the blastocyst hatching stage. Instead, these embryos must be immediately transferred into the uterus.

In addition, transfer of the embryo into the uterus must occur within days 7-9 of embryonic development. However, for embryos which must be subsequently transported for embryo transfer, this timeframe allots for a small transport window. Therefore, embryos are typically frozen following sample collection but prior to genome analysis. This is an ineffective process for two primary reasons. Firstly, the frozen cells may have undesirable genes and therefore time and storage are expended on cells that may be later discarded. Secondly, although having the option to store cells for later use is beneficial, these storage techniques reduce the viability of embryos. Therefore, genotyping techniques which provide results prior to the blastocyst hatching will provide results early enough where the embryo can still be stored or transported for embryo transfer into the uterus. As a result, storage space can be reserved for only the cells with desirable traits and a greater transport window will be provided for those cells which are instead going to be subsequently transferred into the uterus.

Due to the current challenges, we need genotyping techniques that can provide hereditary information while maintaining the viability of the embryo and allowing for subsequent transport and embryo transfer or storage of the embryo via protocols, including but not limited to, freezing and vitrification.

### SUMMARY OF THE INVENTION

The systems and methods of the present disclosure solve these and other issues by providing techniques to perform embryo genotyping using less genetic material. By using less genetic material, the systems and method of the present disclosure can be completed at a lower cost and at a fast enough rate to obtain results prior to blastocyst hatching. In particular, genetic information from the parents of the embryo can be used in a genetic imputation process with a limited genetic sample from the embryo. The systems and methods described herein provide techniques for sample genotyping by low coverage sequencing based on informative single nucleotide polymorphism variants and associated haplotype blocks. The techniques described herein can be applied to a variety of genetic applications, such as to samples of cell-free deoxyribonucleic acid (cfDNA), or any sample for which parental genetic information is known or maintained. The techniques described herein significantly improve the computational efficiency of embryo genotyping, while improving overall viability of embryos.

At least one aspect of the present disclosure is directed to a method for organism genotyping and using genomic data for genotype imputation. The method can be performed, for example, by one or more processors coupled to a non-transitory memory. The method can include maintaining first genetic sequence information of a sire and second genetic sequence information for a dam, the first and second genetic sequence information indicating one or more single nucleotide polymorphisms (SNPs) of interest. The method can include sequencing, based on a skim sequencing technique, a sample of genetic information of a progeny of the sire and the dam. The method can include identifying one or more informative variants based on the first genetic sequence information and the second genetic sequence information. The method can include identifying one or more informative reads in the sequence of the progeny based on the one or more informative variants. The method can include constructing a genotype of the progeny based on the one or more informative reads and the one or more SNPs of interest in the first and second genetic sequence information. In some implementations, the computational genotype imputation can occur after biopsy but prior to blastocyst hatching.

In some implementations, the progeny is an embryo. In some implementations, the method can include terminating the embryo based on the genotype having one or more undesirable genes or variants. In some implementations, the method can include using the embryo for embryo transfer in an in vitro fertilization (IVF) process based on the genotype having one or more desirable genes or variants. In some implementations, the method can include cloning the embryo based on the genotype having one or more desirable genes or variants. In some implementations, the method can include creating a cell line using the embryo based on the genotype having one or more desirable genes or variants. In some implementations, the method can include splitting the embryo based on the genotype of the progeny having one or more desirable genes or variants. In some implementations, the method can include performing a vitrification process on the embryo based on the genotype of the progeny. In some implementations, the method can include selecting the embryo as a future sire or a future dam based on the progeny having one or more desirable genes or variants.

In some implementations, sequencing the sample of the genetic information of the progeny can include sequencing the sample of genetic information of the progeny at a low coverage. Low coverage may correspond to, for example, about 300,000 reads wherein each read comprises a length of 50 base pairs (bp). In various embodiments, low coverage may correspond to about 200,000 or fewer reads wherein each read comprises a length of 25-150 bp, about 300,000 or fewer reads wherein each read comprises a length of 25-150 bp, about 500,000 or fewer reads wherein each read comprises a length of 25-150 bp, or about 1 million or fewer reads wherein each read comprises a length of 25-150 bp. The length of each the reads may be about, for example, 25 bp in length, 50 bp in length, 75 bp in length, 100 bp in length, 125 bp in length, or 150 bp in length. In one embodiment, the number of reads may be 30,000 and the length of each read may be 3,000 bp in length. In one embodiment, the number of reads provides a coverage of the genome of approximately 0.0075× coverage. Other combinations of coverage are also possible, for example but without limitation, 0.004×, 0.0045×, 0.005×, 0.0055×, 0.006×, 0.0065×, 0.007× coverage. Additional combinations of coverage could include, for example, 0.007×-1× coverage and 1×-2× coverage. In some implementations, identifying the one or more informative variants further comprises identifying a first phased region in the first genetic sequence information that is homozygous that corresponds to a second phased region in the second genetic sequence information that is heterozygous. In some implementations, identifying the one or more informative reads further comprises searching the sequence of the progeny to identify matches with the one or more informative variants.

In some implementations, constructing the genotype of the progeny further comprises constructing the genotype of the progeny to include one or more haplotype blocks of the first genetic sequence information or the second genetic sequence information. In some implementations, the genotype of the progeny is constructed further based on a phased haplotype block. In some implementations, the method can include performing a phase cleaning technique over the genotype of the progeny.

At least one other aspect of the present disclosure is directed to a system for embryo genotyping and using genomic data for genotype imputation. The system can include one or more processors coupled to a non-transitory memory. The system can maintain first genetic sequence information of a sire and second genetic sequence information for a dam, the first and second genetic sequence information indicating one or more SNPs of interest. The system can sequence, based on a skim sequencing technique, a sample of genetic information of a progeny of the sire and the dam. The system can identify one or more informative variants based on the first genetic sequence information and the second genetic sequence information. The system can identify one or more informative reads in the sequence of the progeny based on the one or more informative variants. The system can construct a genotype of the progeny based on the one or more informative reads and the one or more SNPs of interest in the first and second genetic sequence information.

In some implementations, the system can sequence the sample of the genetic information of the progeny by performing operations comprising sequencing the sample of genetic information of the progeny at a low coverage corresponding to, for example, about 300,000 reads. In some implementations, the system can identify the one or more informative variants by performing operations comprising identifying a first phased region in the first genetic sequence information that is homozygous that corresponds to a second phased region in the second genetic sequence information that is heterozygous. In some implementations, the system can identify the one or more informative reads by performing operations comprising searching the sequence of the progeny to identify matches with the one or more informative variants.

In some implementations, the system can construct the genotype of the progeny by performing operations comprising constructing the genotype of the progeny to include one or more haplotype blocks of the first genetic sequence information or the second genetic sequence information. In some implementations, the genotype of the progeny is constructed further based on a phased haplotype block. In some implementations, the system can perform a phase cleaning technique over the genotype of the progeny.

At least one other aspect of the present disclosure is directed to a non-transitory computer-readable storage medium having instructions embodied thereon. The instructions, when executed by one or more processors, cause the one or more processors to perform operations. The operations can include maintaining first genetic sequence information of a sire and second genetic sequence information for a dam, the first and second genetic sequence information indicating one or more SNPs of interest. The operations can include sequencing, based on a skim sequencing technique, a sample of genetic information of a progeny of the sire and the dam. The operations can include identifying one or more informative variants based on the first genetic sequence information and the second genetic sequence information. The operations can include identifying one or more informative reads in the sequence of the progeny based on the one or more informative variants. The operations can include constructing a genotype of the progeny based on the one or more informative reads and the one or more SNPs of interest in the first and second genetic sequence information.

In some implementations, the instructions, when executed by the one or more processors, cause the one or more processors to perform further operations including sequencing the sequencing the sample of genetic information of the progeny at a low coverage corresponding to about 300,000 reads. In some implementations, the instructions, when executed by the one or more processors, cause the one or more processors to perform further operations including identifying a first phased region in the first genetic sequence information that is homozygous that corresponds to a second phased region in the second genetic sequence information that is heterozygous. In some implementations, the instructions, when executed by the one or more processors, cause the one or more processors to perform further operations including searching the sequence of the progeny to identify matches with the one or more informative variants.

In some implementations, the instructions, when executed by the one or more processors, cause the one or more processors to perform further operations including constructing the genotype of the progeny to include one or more haplotype blocks of the first genetic sequence information or the second genetic sequence information. In some implementations, the instructions, when executed by the one or more processors, cause the one or more processors to perform further operations including performing a phase cleaning technique over the genotype of the progeny.

In another implementation, the progeny can include, but is not limited to, an embryo. In another implementation, the progeny can be frozen, vitrified, implanted, cloned, split or combinations thereof based on the genotype of the progeny having one or more desirable genes or variants. In yet another implementation, the progeny can be selected as a future sire or a future dam or used to create a cell line based on the genotype of the progeny having one or more desirable genes or variants. In another implementation, the progeny can be terminated based on the genotype of the progeny having one or more undesirable genes or variants.

In another implementation, the sequencing of the sample of genetic information of the progeny which can include, but is not limited to, sequencing, by the one or more processors, the sample of genetic information of the progeny at a low coverage corresponding to about 0.004×-2× coverage. In yet another implementation, the sequencing of the sample of genetic information of the progeny can include, but is not limited to, sequencing, by the one or more processors, the sample of genetic information of the progeny at a low coverage corresponding to about 500,000 or fewer reads with a length of about 25-150 bp.

In another implementation, the identification of one or more informative variants can include, for example, but not limited to, identifying a first phased region in the first genetic sequence information that is homozygous that corresponds to a second phased region in the second genetic sequence information that is heterozygous.

In another implementation, the identification of the one or more informative reads can include, but is not limited to, searching, by the one or more processors, the sequence of the progeny to identify matches with the one or more informative variants.

In another implementation, the constructing of the genotype of the progeny can include, but is not limited to, constructing, by the one or more processors, the genotype of the progeny to include one or more haplotype blocks of the first genetic sequence information or the second genetic sequence information. In another implementation, the genotype of the progeny can be further constructed based on a phased haplotype block.

In yet another implementation, the sample can consist of genetic information extracted from four or fewer cells.

In another implementation, a phase cleaning technique can be performed over the genotype of the progeny. The phase cleaning technique, can include, but is not limited to, correcting phase flipping by executing a median filter to correct incorrect phasing.

In various embodiments, the present teachings include the following non-limiting aspects a system for computational genotype imputation, the system comprising: one or more processors coupled to a non-transitory memory, the one or more processors configured to: maintain first genetic sequence information of a sire and second genetic sequence information for a dam, the first and second genetic sequence information indicating one or more single nucleotide polymorphisms (SNPs) of interest; sequence, based on a skim sequencing technique, a sample of genetic information of a progeny of the sire and the dam; identify one or more informative variants based on the first genetic sequence information and the second genetic sequence information; identify one or more informative reads in the sequence of the progeny based on the one or more informative variants; and construct a genotype of the progeny based on the one or more informative reads and the one or more SNPs of interest in the first and second genetic sequence information. In yet another implementation, the computational genotype imputation can occur after biopsy but prior to blastocyst hatching.

In another implementation, the one or more processors can be further configured to sequence the sample of the genetic information of the progeny by performing operations comprising sequencing the sample of genetic information of the progeny at a low coverage corresponding to about 500,000 or fewer reads with a length of about 25-150 bp. In yet another implementation, the one or more processors can be further configured to sequence the sample of the genetic information of the progeny by performing operations comprising sequencing the sample of genetic information of the progeny at a low coverage corresponding to about 0.004×-2× coverage.

In another implementation, the one or more processors can be further configured to identify the one or more informative variants by performing operations comprising identifying a first phased region in the first genetic sequence information that is homozygous that corresponds to a second phased region in the second genetic sequence information that is heterozygous. In yet another implementation, the one or more processors can be further configured to identify the one or more informative reads by performing operations comprising searching the sequence of the progeny to identify matches with the one or more informative variants.

In another implementation, the one or more processors can be further configured to construct the genotype of the progeny by performing operations comprising constructing the genotype of the progeny to include one or more haplotype blocks of the first genetic sequence information or the second genetic sequence information. In yet another implementation, the one or more processors can be further configured to perform a phase cleaning technique over the genotype of the progeny.

In various embodiments, the present teachings include the following non-limiting aspects a non-transitory computer-readable storage medium having instructions embodied thereon, the instructions, when executed by one or more processors, cause the one or more processors to perform operations of constructing a genotype of a progeny embryo based on a sample of genetic information from the progeny embryo, the operations comprising: maintaining first genetic sequence information of a sire and second genetic sequence information for a dam, the first and second genetic sequence information indicating one or more single nucleotide polymorphisms (SNPs) of interest; sequencing, based on a skim sequencing technique, a sample of genetic information of a progeny embryo of the sire and the dam; identifying one or more informative variants based on the first genetic sequence information and the second genetic sequence information; identifying one or more informative reads in the sequence of the progeny embryo based on the one or more informative variants; and constructing a genotype of the progeny based on the one or more informative reads and the one or more SNPs of interest in the first and second genetic sequence information. In yet another implementation, constructing a genotype of the progeny can occur after biopsy but prior to blastocyst hatching.

In another implementation, the instructions, when executed by the one or more processors, cause the one or more processors to perform further operations comprising sequencing the sequencing the sample of genetic information of the progeny at a low coverage corresponding to about 500,000 or fewer reads with a length of about 25-150 bp.

In yet another implementation, the instructions, when executed by the one or more processors, cause the one or more processors to perform further operations comprising sequencing the sequencing the sample of genetic information of the progeny at a low coverage corresponding to about 300,000 or fewer reads with a length of about 25-150 bp.

In another implementation, the instructions, when executed by the one or more processors, cause the one or more processors to perform further operations comprising identifying a first phased region in the first genetic sequence information that is homozygous that corresponds to a second phased region in the second genetic sequence information that is heterozygous.

In another implementation, the instructions, when executed by the one or more processors, cause the one or more processors to perform further operations comprising searching the sequence of the progeny to identify matches with the one or more informative variants.

In another implementation, the instructions, when executed by the one or more processors, cause the one or more processors to perform further operations comprising constructing the genotype of the progeny to include one or more haplotype blocks of the first genetic sequence information or the second genetic sequence information.

In another implementation, the instructions, when executed by the one or more processors, cause the one or more processors to perform further operations comprising performing a phase cleaning technique over the genotype of the progeny.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect. As used in the specification and in the claims, the singular form of ‘a’, ‘an’, and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates an example system for embryo genotyping and using genomic data for genotype imputation, in accordance with one or more implementations;

FIGS. 2A and 2B illustrate diagrams of an example genotype imputation process, in accordance with one or more implementations;

FIG. 3 illustrates an example flow diagram of a method for embryo genotyping and using genomic data for genotype imputation;

FIG. 4 illustrates the general architecture of an illustrative computer system that may be employed to implement any of the computers discussed herein.

### DETAILED DESCRIPTION

Below are detailed descriptions of various concepts related to, and implementations of, techniques, approaches, methods, apparatuses, and systems for embryo genotyping and using genomic data for genotype imputation. The various concepts introduced above and discussed in greater detail below may be implemented in any of numerous ways, as the described concepts are not limited to any particular manner of implementation. Examples of specific implementations and applications are provided primarily for illustrative purposes.

The systems and methods of this technical solution provide techniques to accurately and rapidly perform embryo genotyping prior to blastocyst hatching. To do so, imputation of genetic information is performed based on genetic information gathered from the parents of the embryo. These techniques may be implemented for any purpose for which genetic information may be used, such as determining whether a particular embryo of an animal will be viable in the long-term (e.g., free of genetic issues). Other genetic sequencing techniques require an abundance of genetic information, the extraction of which would compromise the viability of the embryo and the analysis would extend beyond the blastocyst hatching period. In addition, techniques that utilize minimal genetic information would typically omit genetic information that is relevant to determining whether the embryo will be viable, or have certain genetic characteristics. However, the systems and methods described herein solve these issues by utilizing a minimal amount of genetic information from an embryo early in development in a genetic imputation process, which may be used to accurately determine large segments of genetic information of the offspring by using genetic information from the parents. Also, unlike other approaches, the present techniques allow for rapid genotyping. The present techniques can therefore be used to genotype embryos prior to blastocyst hatching which allows for subsequent transport and implantation of the embryo or storage of the embryo via protocols, including but not limited to, freezing and vitrification.

Alternatively, DNA obtained from an embryo transfer, maturation, IVF, or other media may be used. This type of DNA source may be referred to as “cell-free DNA.” As used herein, “cell-free DNA” refers to embryonic DNA that accumulates in the medium of an embryo culture and that is accessible without performing a biopsy on the embryo. Preferably, cell free-DNA is obtained without the need to manipulate or disrupt embryonic cells to obtain the DNA. Skilled artisans will recognize that the medium may still contain cellular debris that has been naturally shed into the culture, and that samples of cell-free DNA may include such cellular debris. The cell-free DNA may be from many different sources, including micro or macro vesicles released from the embryo during in vitro culture.

As used herein, the terms “genotyping” and “genotype” refer, respectively, to methods and the results of methods to determine information about a genome. The information obtained can identify, for example, a specific single nucleotide polymorphism (SNP) or other genetic marker, any collection of genetic markers (including, for example, chip-based assay arrays) or any lengths of any number of genetic sequences measured from a genome. Genotype may include both a measured genotype, which is the genotype as measured or read and which may include read errors, sequence gaps, or other errors, and may also include an actual genotype, which is the sequence of nucleotides in the genome or DNA.

These and other improvements are described in detail herein below.

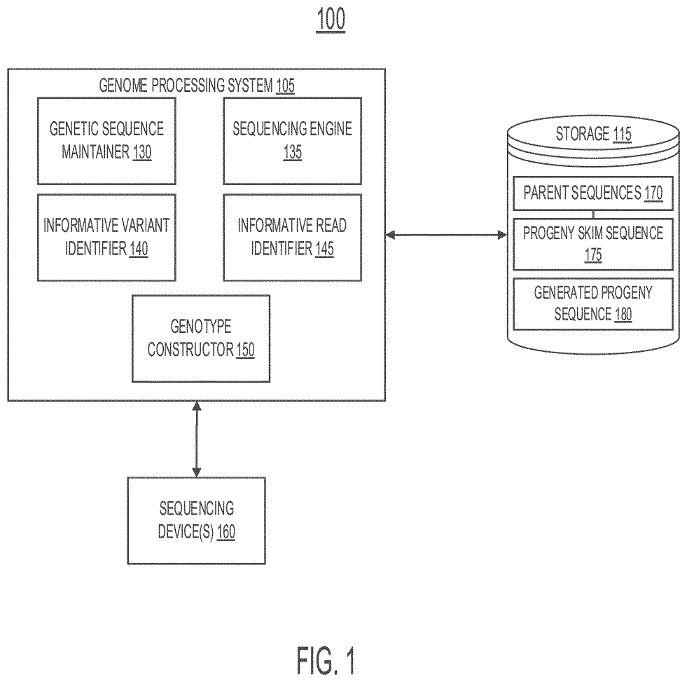

Referring now to FIG. 1, illustrated is a block diagram of an example system 100 for embryo genotyping and using genomic data for genotype imputation, in accordance with one or more implementations. The system 100 can include at least one genome processing system 105, at least storage 115, and one or more sequencing devices 160. The genome processing system 105 can include at least one genetic sequence maintainer 130, at least one sequencing engine 135, at least one informative variant identifier 140, at least one informative read identifier 145, and at least one genotype constructor 150. The storage 115 can maintain one or more parent sequences, at least one progeny skim sequence 175, and at least one generated progeny sequence 180.

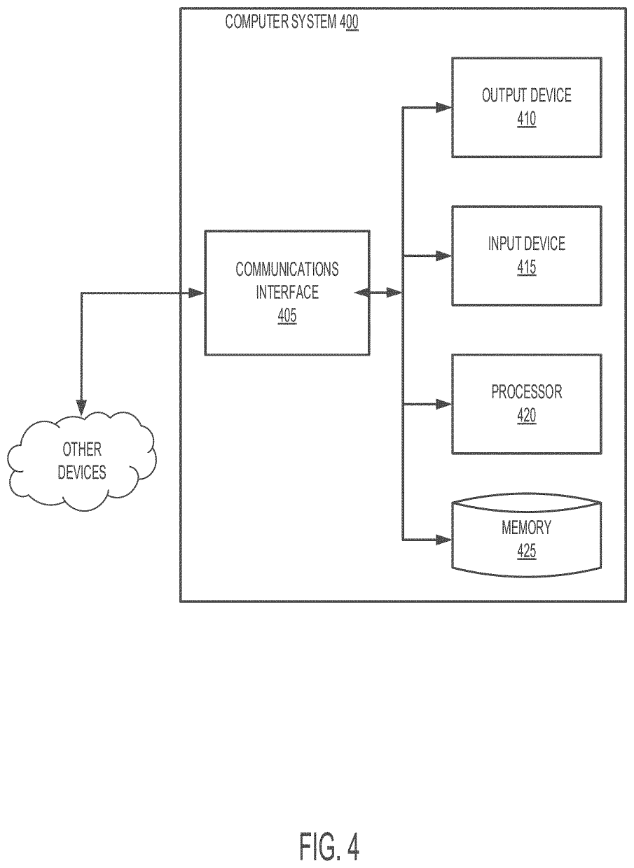

Each of the components (e.g., the genome processing system 105, the genetic sequence maintainer 130, the sequencing engine 135, the informative variant identifier 140, the informative read identifier 145, the genotype constructor 150, the storage 115, etc.) of the system 100 can be implemented using the hardware components or a combination of software with the hardware components of a computing system (e.g., computing system 400, the genome processing system 105, any other computing system described herein, etc.) detailed in connection with FIG. 4. Each of the components of the genome processing system 105 can perform the functionalities detailed herein.

The genome processing system 105 can include at least one processor and a memory (e.g., a processing circuit). The memory can store processor-executable instructions that, when executed by processor, cause the processor to perform one or more of the operations described herein. The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The genome processing system 105 can include one or more computing devices or servers that can perform various functions as described herein. The genome processing system 105 can include any or all of the components and perform any or all of the functions of the computer system 400 described herein in conjunction with FIG. 4.

The one or more sequencing devices 160 can be any type of sequencing device or system that is capable of extracting genetic sequence data, such as the parent sequences 170 or the progeny skim sequence 175, from genetic material. For example, the one or more sequencing devices may be sequencing devices or systems that can perform high coverage or low coverage whole genome sequencing (WGS) on genetic material. In some implementations, at least one of the sequencing devices 160 can be utilized to perform a skim sequencing technique (sometimes referred to as “skim sequencing,” “genome skimming,” or “shallow sequencing”). The sequencing devices 160 can perform such sequencing techniques on any type of genetic material, including any type of deoxyribonucleic acid (DNA) or ribonucleic acid (RNA). Some examples of sequencing devices can include, but are not limited to, Sanger sequencing devices, shotgun sequencing devices, single-molecule real-time sequencing devices, ion semiconductor sequencing devices, sequencing by synthesis devices, combinatorial probe anchor synthesis devices, sequencing by ligation devices, nanopore sequencing devices, and chain termination sequencing devices, among others.

The sequencing devices 160 can be utilized to perform high coverage whole genome sequencing (WGS) or low coverage WGS such as skim sequencing. High coverage whole genome sequencing can be performed, for example, to extract all of the genetic information from the parents, which may then be used to “fill in” gaps identified in genes of interest in the progeny, which can then be used to determine genetic properties of the embryo as a whole, such as the embryo's overall viability (e.g., likelihood to survive to adulthood). In addition, the techniques described herein may be used to identify desirable or undesirable traits of an organism, such as disease resistance, carcass characteristics, or feed efficiency, among others. These techniques may also be used to avoid genetic defects, including negative effects from SNPs such as large deletions of genetic information. In some implementations, the sequencing devices 160 can be used to perform high coverage WGS on genetic information of the parents (e.g., a sire and a dam) of a progeny embryo of interest. Generally, high coverage WGS is the process of determining the entirety, or nearly the entirety, of the DNA sequence of an organism's genome. This can include sequencing both the chromosomal DNA and the mitochondrial DNA of the organism (e.g., the parents) to produce a genetic code sequence (e.g., the parent sequences 170). In addition, one or more of the sequencing devices 160 can perform skim sequencing on limited amounts of genetic information (e.g., a few cells from a progeny embryo).

In contrast to high coverage WGS, low coverage WGS such as skim sequencing uses low-pass, shallow sequencing to generate fragments of DNA, which may be referred to as “skims” or “genome skims.” Generally, skim sequences are low-coverage sequencing techniques that are significantly less expensive to sequence and much faster to process than high-coverage whole genome sequencing. Skim sequencing protocols also allow for rapid genotyping with biopsy to genotyping protocols able to be completed in under 48 hours. This rapid genotyping provides results prior to the blastocyst hatching stage and therefore the genotyped embryo can be subsequently implanted, frozen or vitrified. It should be understood that quicker results are a client/customer demand in most industries and therefore the methods and system disclosed herein provide that additional benefit beyond the one described in embryo analysis.

The sequencing devices 160 can communicate with the genome processing system 105 or the storage 115, for example, via one or more electronic communication interfaces (e.g., an appropriate computer communication bus or interface, an electronic computer network, etc.). The results of any genome sequencing operation performed by the sequencing devices 160 can be stored in the memory of the genome processing system 105 or in the storage 115. In some implementations, the genome processing system 105 can receive the sequenced genetic information from the sequencing devices 160 and store the resulting sequences (e.g., the parent sequences 170, the progeny skim sequence 175, etc.) in one or more indexed data structures in the storage 115. The indexed data structures of the various genetic sequences can then be accessed by the genome processing system 105 to perform the techniques described herein.

The storage 115 can be a computer memory device or database that stores or maintains any of the information described herein. The storage 115 can maintain one or more data structures, which may contain, index, or otherwise store each of the values, pluralities, sets, variables, vectors, data structures, or thresholds described herein. The storage 115 can be accessed using one or more memory addresses, index values, or identifiers of any item, structure, or region maintained in the storage 115. The storage 115 can be accessed by the components of the genome processing system 105, or any other computing device described herein, via a network or other suitable communication interface. In some implementations, the storage 115 can be internal to the genome processing system 105. In some implementations, the storage 115 can exist external to the genome processing system 105, and may be accessed via a network or suitable electronic communication interface. The storage 115 can be distributed across many different computer systems or storage elements, and may be accessed via a network or a suitable computer bus interface. The genome processing system 105 can store, in one or more regions of the memory of the genome processing system 105, or in the storage 115, the results of any or all computations, determinations, selections, identifications, generations, constructions, or calculations in one or more data structures indexed or identified with appropriate values. Any or all values stored in the storage 115 may be accessed by any computing device described herein, such as the genome processing system 105, to perform any of the functionalities or functions described herein.

The storage 115 can store or maintain one or more parent sequences 170, which may correspond to a genetic sequences of parents of an embryo of interest. The parent sequences 170 can be, for example, the fully sequenced genome of each parent of an embryo of interest. The parent sequences can be produced using one or more sequencing devices 160. The parent sequences 170 comprise high coverage WGS sequence data generated by the sequencing devices 160, identifiers of SNPs of interest extracted using any type of chromosome-level phasing techniques (e.g., chip phasing, sequencing high coverage WGS data to allow for contiguous phase blocks, etc.), and informative markers identified by the genome processing system 105 of FIG. 1. In some implementations, the parent sequences 170 can include any feature that is identifiable and usable with a haplotype block. For example, if a read indicated that a deletion was present, and data showed the deletion was present on a sire, then that portion of the genetic information may be informative and therefore usable as part of the parent sequences. The parent sequences 170 can include indications of duplications, deletions, inversions, insertion-deletion mutations (indels), or short tandem repeats, among others. The parent sequences 170 of the parents can be sequenced to identify one or more SNPs of interest. SNPs are germline substitutions of a single nucleotide at a specific position of the genome. SNPs of interest can be selected by a user, for example, to be known genes that affect embryo viability or overall organism health. The SNPs of interest may be specific to the type of organism. As part of the sequencing techniques used to create the parent sequences 170, the genetic information can be applied to a chip SNP phasing technique to extract portions of genetic information that correspond to predetermined regions of the genome of the organism. For example, the SNPs can be interrogated simultaneously on a high-density SNP array that includes probes for predetermined SNPs of interest.

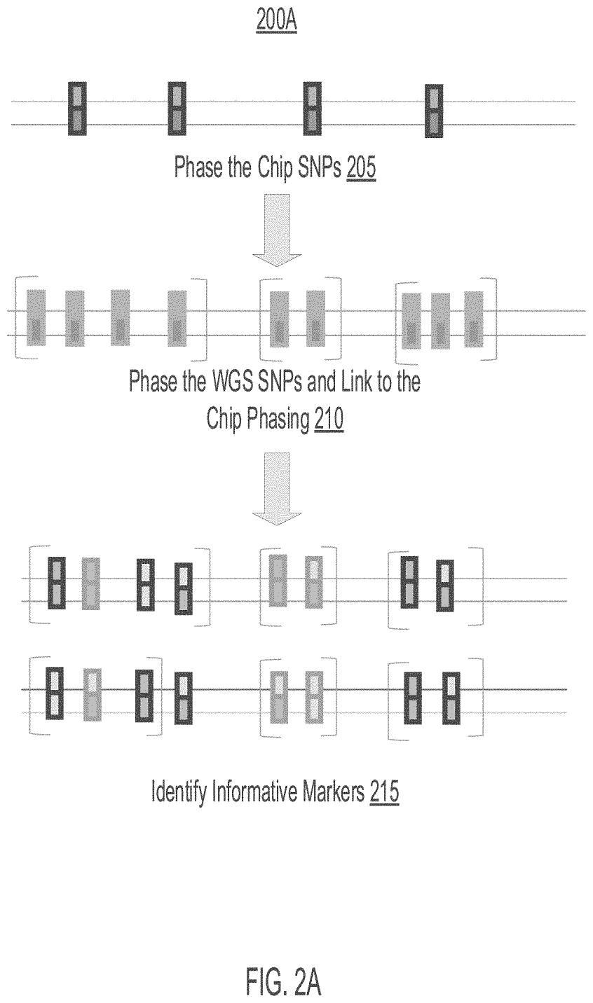

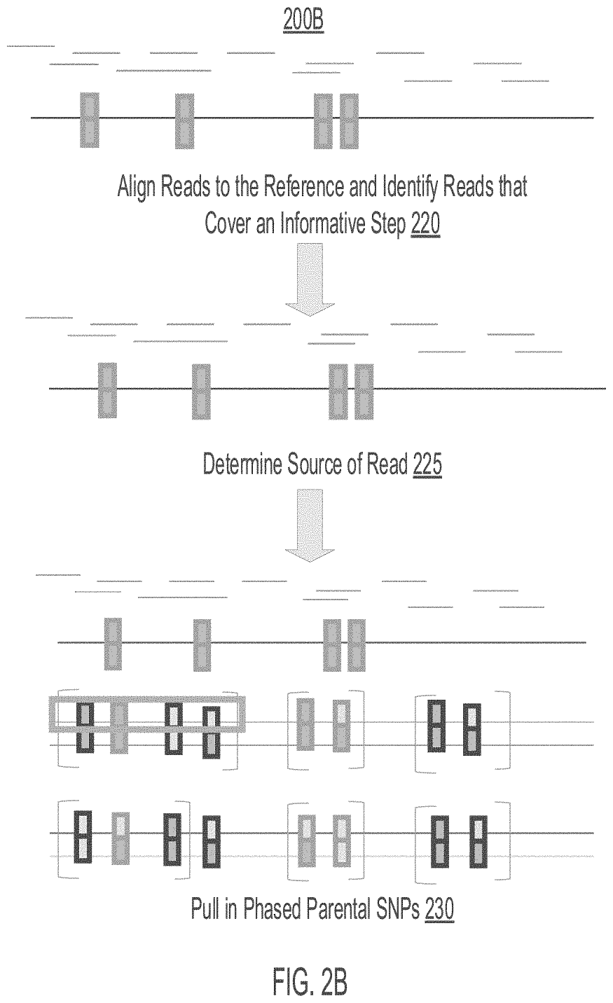

An overview of the pre-work performed as part of assembling the parent sequences 170 is shown in FIG. 2A. Referring briefly to FIG. 2A, depicted is an example pre-work process performed to generate the parent sequences 170. As shown in FIG. 2A, at step 205, the chip SNPs of the parent genetic information are first phased according to the SNPs of interest. For example, a SNP array (sometimes referred to herein as a “chip”) may include a number of high-density probes which are configured to bind to and identify the presence of certain genotypes, genes, or alleles. The probes can be pre-configured to bind to a predetermined genotype, gene, or allele. In some implementations, redundancy can be introduced to reduce overall error frequency in the chip. Once the chip SNPs are phased, the high coverage WGS sequencing information in the parent sequences 170 can be linked to the chip phasing. For example, the information in the parent chip data can be used to identify predetermined regions in the high coverage WGS sequence of both the sire and the dam of the progeny undergoing analysis. Once these regions have been linked, informative markers in the parent sequences 170 can be identified. Further details of these steps are described in detail herein below in connection with the operations of the genome processing system 105 of FIG. 1.

Referring back now to FIG. 1, the storage 115 can store or maintain a progeny skim sequence 175 of a progeny embryo undergoing analysis, and therefore can be stored in association with corresponding parent sequences 170 of the sire and the dam of the progeny. The progeny skim sequence 175 can be generated from a limited amount of genetic material of the progeny embryo (e.g., a few cells). Skim sequencing can be performed by one or more of the sequencing devices 160, and can be sequenced at a low coverage corresponding to about 300,000 reads, for example, where each read comprises a length of 50 bp. However, it should be understood that other low-coverage amounts are also possible, and that 300,000 reads, with each having a length of 50 bp, is merely provided for example purposes. For example, the number of reads may be 500,000 or fewer or 200,000 or fewer, and each read may include 25-150 bp. In some implementations, the number of reads may be 30,000, and the length of each read may be 3,000 bp in length. The number of reads may provide a coverage of the genome of approximately 0.0075× coverage. Other combinations of coverage are also possible, for example but without limitation, 0.004×, 0.0045×, 0.005×, 0.0055×, 0.006×, 0.0065×, 0.007× coverage. Additional combinations of coverage could include, for example, 0.007×-1× coverage and 1×-2× coverage. In some implementations, the information in the progeny skim sequence 175 can be filtered to the genetic information corresponding to the SNPs of interest (e.g., at predetermined positions in the genome, etc.). The progeny skim sequence can be used by the genome processing system 105 as part of the techniques described herein to generate the generated progeny sequence 180.

The storage 115 can store or maintain the generated progeny sequence 180, which is generated by the genome processing system 105 as described herein below. The generated progeny sequence 180 may include additional genetic information derived from the parental genomes of the progeny, by executing the algorithms described herein over the progeny skim sequence 175. To do so, the genome processing system 105 can perform operations similar to those described in connection with FIG. 2B. Referring briefly to FIG. 2B, depicted is an example process 200B to genotype the progeny by generating a generated progeny sequence (e.g., the generated progeny sequence 180 of FIG. 1). The process shown in FIG. 2B can follow the pre-work shown in FIG. 2A. As shown in FIG. 2B, the process on the progeny skim sequence (e.g., the progeny skim sequence 175 of FIG. 1) begins at step 220 by aligning the reads to the reference sequences (e.g., the parent sequences 170 of FIG. 1). Recall that in the previous step 215, informative markers (and the SNPs corresponding thereto) were identified in the parent sequences. Upon aligning the skim sequence of the progeny to the parent sequence, portions of the skim sequence (reads) that correspond to an informative SNP can be identified (e.g., the portions of the genetic information that match) in step 220. Once the read has been matched, the source of the read (e.g., which parent and which chromosome) can be identified in step 225, for example, by performing a matching look up in the parent sequences. Once the source has been identified, the full phased parent SNPs can be pulled in from the parents to fill out the gaps in the skim sequence of the progeny to create a generated progeny sequence (e.g., the generated progeny sequence 180 of FIG. 1) at step 230. Further details of this process are described in connection with the genome processing system 105 shown in FIG. 1. Referring back to FIG. 1, the generated progeny sequence 180 can include genetic information that is generated as part of a genotype imputation technique (e.g., using the phased SNPs of the parental sequences 170). The generated progeny sequence 180 can be generated to include the genotypes of genes of interest, such as genes that correspond to the viability of the embryo being analyzed. The presence of status of particular genes of interest in the generated progeny sequence 180 can be used to determine the viability of the embryo undergoing analysis.

Therefore, using the techniques described herein, the overall viability of an embryo can be determined with very limited genetic information, and prior to significantly investing in the growth of that embryo. This is an improvement over other genotyping techniques, because other genotyping techniques cannot produce results that are as accurate with as limited genetic information. In addition, the present techniques are a rapid genotyping process, and therefore allow for embryo genotyping can be completed prior to blastocyst hatching which allows for subsequent transport and implantation of the embryo or storage of the embryo via protocols, including but not limited to, freezing and vitrification. Other techniques rely solely on high coverage WGS sequencing information, and therefore require significantly longer processing time than the techniques described herein which would require samples to be stored prior to obtaining genome analysis results or the embryo would have to implanted within a small timeframe. Therefore, the systems and methods of the present disclosure provide significant improvements over other embryo sequencing systems.

Referring now to the operations of the genome processing system 105, the genetic sequence maintainer 130 can maintain genetic sequence information of the parents of a progeny of interest. The present techniques may utilize genetic sequence information from the parents of an embryo (sometimes referred to herein as a progeny) to fill in gaps in the genome of the embryo. To do so, the genetic sequence maintainer 130 may communicate with one or more sequencing devices 160, or with external computing devices (not pictured) to retrieve, store, and access the parent sequences 170. The parents of the progeny embryo under analysis may be sequenced at a sufficient depth to genotype the parents genome-wide (e.g., 20× to 30× for sequencing platforms such as Illumina, etc.). The first and second genetic sequence information (e.g., sequences from each parent in the parent sequences 170) may indicate one or more SNPs of interest, which may be phased using a SNP array (e.g., which may be, or be a part of, one of the one or more sequencing devices 160). Other genetic features may also be used to identify informative variants, such as copy number variations (CNVs), deletions, indels, insertions, inversions, and short tandem repeat polymorphisms, among others. The genetic information of the parents can be phased using a genome phasing technique to phase each of the identified SNPs of interest (e.g., identify the chromosome and parent from each of the parental sequences 170). The variants of the sequences of each parent can be phased from the high coverage WGS sequences of each parent. Identifiers and attributes of the phased SNPs and the phased variants can then be stored in association with the corresponding parent sequences 170 in the storage 115. As described herein above, the SNPs of interest may correspond to predetermined genotypes, such as genotypes that correspond to the viability of an embryo. The SNPs of interest can then be used in a genotype imputation process to determine the viability of an embryo using limited genetic information.

The sequencing engine 135 can sequence a sample of genetic information (e.g., progeny skim sequence 175) of the progeny of the sire and the dam, which provided the parent sequences 170. The sequencing engine 135 can sequence the genetic information via one or more of the sequencing devices 160, any of which may utilize a skim sequencing technique. As described herein above, a skim sequencing technique is a technique that provides low coverage reads of genetic information using a very small sample size (e.g., only a few cells). It is important that the technique supports extremely low input DNA amounts, because genotyping techniques that require more genetic material would require a sample size that could potentially damage the viability of the embryo. By using a sample size as small as a few cells, the techniques described herein can be performed on embryos very early in development. Therefore, the present techniques can be performed on embryos prior to implantation and without freezing or vitrification, in a cost effective manner. The sequencing engine 135 may communicate with one or more sequencing devices 160 that perform skim sequencing on a sample of the progeny embryo, and retrieve genetic sequence data and store the genetic sequence data as the progeny skim sequence 175. Sequencing the genetic information of the progeny can be performed at a low coverage corresponding to, for example, about 300,000 reads. However, other numbers and lengths of reads may be utilized, as described herein. In some implementations, the sequencing engine 135 may filter the progeny skim sequence 175 to only information corresponding to the SNPs of interest (e.g., corresponding genetic information). To do so, the sequencing engine 135 may align the progeny skim sequence with the high coverage WGS sequence data of the parents stored as part of the parent sequences 170. Portions of the progeny skim sequence that are not related to the SNPs of interest (e.g., parts of different haplotype blocks that do not include SNPs of interest, etc.) may be discarded. Low-coverage skim sequencing is faster than whole-genome sequencing processes performed in other embryo genotyping techniques. By utilizing skim sequencing and the genotype imputation techniques described herein, the processes significantly improve the performance of embryo genotyping.

The informative variant identifier 140 can identify one or more informative variants based on the genetic sequence information of the sire and the genetic sequence information of the dam (e.g., the parent sequences 170). To identify an informative variant, the informative variant identifier 140 can identify a first phased region in the genetic information (e.g., the parent sequences of one parent that is homozygous that corresponds to a second phased region in the genetic sequence information of the other parent that is heterozygous. Generally, variants are changes in a DNA sequence. To identify informative variants, or variants in genetic information that identify from which parent the progeny inherited a corresponding haplotype block, the informative variant identifier 140 can scan through the SNPs of interest in each parent to identify variants. If a variant is detected (e.g., one parent is homozygous and the other parent is heterozygous, etc.), the informative variant identifier 140 can store a flag identifying the corresponding SNP and the corresponding location in the genome of the organism at which the variant was identified. This information can be stored in the memory of the genome processing system 105, or as part of the parent sequences 170.

The informative read identifier 145 can identify one or more informative reads in the sequence of the progeny based on the one or more informative variants identified from the parent sequences 170 generated from the parents of the progeny. To do so, the informative read identifier 145 can search the progeny skim sequence 175 to identify matches with the one or more informative variants. As described above, each informative variant is a genetic variation between each of the parents. Therefore, a SNP of interest in the progeny skim sequence that is present in a haplotype block that corresponds (e.g., is at a corresponding position in the genome of the organism) to an informative variant can be used to identify the parent that provided the haplotype block to the progeny. In other words, searching through the skim sequence and identifying genetic information that corresponds to an informative variant can be used to identify regions of genetic information in the progeny genome that will match a corresponding region in either parent. Therefore, the reads of interest can be any read in the progeny skim sequence 175 that matches an identified informative variant. The informative read SNP in the progeny skim sequence 175 can then be compared to corresponding SNPs in each of the parents in the parent sequences 170. If a match between either parent is identified, the informative read identifier 145 can store an association between the corresponding haplotype block in the parent and the informative read identified in progeny skim sequence 175. These associations can then be used to reconstruct the genome of the embryo through imputation.

The genotype constructor 150 can construct the generated progeny sequence 180 (e.g., the progeny genome) based on the one or more informative reads and the one or more SNPs of interest in the first and second genetic sequence information. To do so, the genotype constructor can scan through each of the identified informative reads in the progeny skim sequence 175 and extract the corresponding haplotype blocks identified by the informative read identifier 145 from each parent. For example, if an informative read is identified as corresponding to the sire, the genotype constructor 150 can extract the haplotype block that includes the genome location of the informative read from the genome sequence of the sire (e.g., stored in the parent sequences 170). Likewise, if an informative read is identified as corresponding to the dam, the genotype constructor 150 can extract the haplotype block that includes the genome location of the informative read from the genome sequence of the dam (e.g., stored in the parent sequences 170). In some implementations, the genotype constructor 150 can utilize the FImpute algorithm to fill in additional gaps (e.g., regions of the progeny that are unsequenced) in the progeny skim sequence 175.

The genotype constructor 150 can perform the FImpute algorithm (e.g., which may utilize many parent sequences 170 from the species of the organism) following the extraction of the corresponding haplotype blocks from the parent sequences 170, which are then populated at corresponding positions in the generated progeny sequence 180. However, in some cases, gaps in the generated progeny sequence may remain, which may be populated using the FImpute algorithm and the parent sequences 170 (or other genetic sequences of other members of the same species, which may be livestock). When implementing the FImpute algorithm, the genotype constructor 150 can utilize an overlapping sliding window to identify haplotype similarity between reference genetic sequences and the generated progeny sequence 180. In some implementations, the genotype constructor 150 can perform a phase cleaning technique over the generated progeny sequence 180. The generated progeny sequence 180 is then stored in the storage 115 in association with an identifier of the embryo. Using the genotype information in the generated progeny sequence 180, the viability of the embryo can be determined. For example, the status of predetermined genes in the generated progeny sequence 180 can identify genetic defects in the progeny, which if present may indicate that the progeny embryo is not viable. The genotype constructor 150 may scan through the generated progeny sequence 180 to identify the status of such predetermined genes, and compare the statuses to a predetermined healthy baseline for each gene. If the genes match the predetermined healthy baseline, then the embryo can be identified as viable. If not, then the embryo can be identified as not viable.

After constructing the progeny sequences 180, one or more desirable or undesirable genes or traits can be identified in the progeny sequence 180 by comparing predetermined genetic locations in the progeny sequence 180 with known genes. These known genes may be maintained in one or more lookup tables identified by organism type. The lookup tables can include one or more desirable or undesirable genes, their expected location in the progeny sequence 180, and any potential traits associated with any status of each gene. This lookup table can be accessed and used to identify whether the progeny sequence 180 includes one or more indicators of desirable or undesirable traits. Identifiers of these desirable or undesirable traits, when detected in the progeny sequence 180, may be presented in a user interface on a display device. In some implementations, the lookup tables can include information related to genetic variations that implicates changes in a genomic index used as a metric or score, such as the NetMerit genetic index score. The genome processing system 105 can utilize the generated progeny sequence 180 to calculate the NetMerit score for the progeny and can also utilize the generated progeny sequences 180 to calculate or determine any other suitable or applicable metric or score. For example, the NetMerit score can factor in the additive effect of small variants in genetic information of the progeny. The information used to calculate the NetMerit score can be in linkage with causative genetic variation.

The presence of desirable or undesirable traits can then be used to determine a course of action for the progeny. For example, if the progeny is an embryo, the embryo may be selected for termination if the progeny sequence 180 includes one or more undesirable traits. Similarly, if the progeny genotype 180 includes desirable traits, the embryo may be selected for embryo transfer in an in vitro fertilization (IVF) process, a cloning process, or an embryo splitting process. In some implementations, a cell line may be created using the embryo if the embryo includes one or more desirable traits. In some implementations, the embryo may be subjected to a vitrification process if the embryo includes one or more desirable traits, or the embryo may be selected as a future sire or a future dam. Similarly, the genome processing system 105 may provide one or more recommendation messages in response to detecting one or more desirable or undesirable traits in the progeny sequence 180. The recommendations can include any of the foregoing courses of action for the progeny. The genome processing system 105 may rank embryos according to their respective scores. The ranking of embryos may be used to select which embryos to implant in limited surrogate carriers.

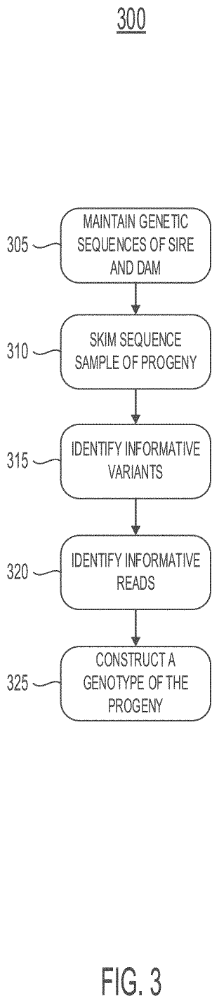

Referring now to FIG. 3, depicted is an illustrative flow diagram of a method 300 for embryo genotyping and using genomic data for genotype imputation. The method 300 can be executed, performed, or otherwise carried out by the genome processing system 105, the computer system 300 described herein in conjunction with FIG. 3, or any other computing devices described herein. In brief overview, the genome processing system (e.g., the genome processing system 105, etc.) can maintain genetic sequences of a sire and a dam (e.g., the parent sequences 170) of a progeny (STEP 305), skim sequence (e.g., the progeny skim sequence 1750 a sample of the progeny (STEP 310), identify informative variants (STEP 315), identify informative reads (STEP 320), and construct a genotype (e.g., the progeny sequence 180) of the progeny (STEP 325).

In further detail, at step 305, the genome processing system can maintain first genetic sequence information of a sire and second genetic sequence information for a dam. The first and second genetic sequence information may indicate one or more single nucleotide polymorphisms of interest. To do so, the genome processing system may communicate with or more sequencing devices (e.g., the sequencing devices 160), or with external computing devices (not pictured) to retrieve, store, and access the parent sequences. The parents of the progeny embryo under analysis may be sequenced at a sufficient depth to genotype the parents genome-wide (e.g., 20× to 30× for sequencing platforms such as Illumina, etc.). The first and second genetic sequence information (e.g., sequences from each parent in the parent sequences) may indicate one or more SNPs of interest, which may be phased using a SNP array (e.g., which may be, or be a part of, one of the one or more sequencing devices). The genetic information of the parents can be phased using a genome phasing technique to phase each of the identified SNPs of interest (e.g., identify the chromosome and parent from each of the parental sequences). The variants of the sequences of each parent can be phased from the high coverage WGS sequences of each parent. Identifiers and attributes of the phased SNPs and the phased variants can then be stored in association with the corresponding parent sequences in memory (e.g., the storage 115). As described herein above, the SNPs of interest may correspond to predetermined genotypes, such as genotypes that correspond to the viability of an embryo. The SNPs of interest can then be used in a genotype imputation process to determine the viability of an embryo using limited genetic information.

At step 310, the genome processing system can sequence, based on a skim sequencing technique, a sample of genetic information of a progeny of the sire and the dam. The genome processing system can sequence the genetic information via one or more of the sequencing devices 160, any of which may utilize a skim sequencing technique. As described herein above, a skim sequencing technique is a technique that provides low coverage reads of genetic information using a very small sample size (e.g., only a few cells). It is important that a low-coverage sequencing technique be used, because skim sequencing is significantly faster, but provides lower coverage, than other sequencing techniques. This accelerated timeframe for embryo sequencing allows for embryos to be sequenced and genotyped without requiring freezing or vitrification. By using a sample size as small as a few cells, the techniques described herein can be performed on embryos very early in development. The genome processing system may communicate with one or more sequencing devices 160 that perform skim sequencing on a sample of the progeny embryo, and retrieve genetic sequence data and store the genetic sequence data as a progeny skim sequence (e.g., the progeny skim sequence 175). Sequencing the genetic information of the progeny can be performed at a low coverage corresponding to about 300,000 reads, or any other number of reads at various lengths, as described herein. In some implementations, the genome processing system may filter the progeny skim sequence to only information corresponding to the SNPs of interest (e.g., corresponding genetic information). To do so, the genome processing system may align the progeny skim sequence with the high coverage WGS sequence data of the parents stored as part of one or more parent sequences (e.g., the parent sequences 170). Portions of the progeny skim sequence that are not related to the SNPs of interest (e.g., parts of different haplotype blocks that do not include SNPs of interest, etc.) may be discarded. By utilizing skim sequencing and the genotype imputation techniques described herein, the processes significantly improve the performance of embryo genotyping.

At step 315, the genome processing system can identify one or more informative variants based on the first genetic sequence information and the second genetic sequence information (e.g., the parent sequences 170 of the parents of the embryo under analysis). To identify an informative variant, the genome processing system can identify a first phased region in the genetic information (e.g., the parent sequences of one parent that is homozygous that corresponds to a second phased region in the genetic sequence information of the other parent that is heterozygous). Generally, variants are changes in a DNA sequence. To identify informative variants, or variants in genetic information that identify from which parent the progeny inherited a corresponding haplotype block, the genome processing system can scan through the SNPs of interest in each parent to identify variants. If a variant is detected (e.g., one parent is homozygous and the other parent is heterozygous, etc.), the genome processing system can store a flag identifying the corresponding SNP and the corresponding location in the genome of the organism at which the variant was identified. This information can be stored in the memory of the genome processing system, or as part of the parent sequences.

At step 320, the genome processing system can identify, by the one or more processors, one or more informative reads in the sequence of the progeny based on the one or more informative variants. To do so, the genome processing system can search the progeny skim sequence to identify matches with the one or more informative variants. As described above, each informative variant is a genetic variation between each of the parents. Therefore, a SNP of interest in the progeny skim sequence that is present in a haplotype block that corresponds (e.g., is at a corresponding position in the genome of the organism) to an informative variant can be used to identify the parent that provided the haplotype block to the progeny. In other words, searching through the skim sequence and identifying genetic information that corresponds to an informative variant can be used to identify regions of genetic information in the progeny genome that will match a corresponding region in either parent. Therefore, the reads of interest can be any read in the progeny skim sequence that matches an identified informative variant. The informative read SNP in the progeny skim sequence can then be compared to corresponding SNPs in each of the parents in the parent sequences. If a match between either parent is identified, the genome processing system can store an association between the corresponding haplotype block in the parent and the informative read identified in progeny skim sequence. These associations can then be used to reconstruct the genome of the embryo through imputation.

At step 325, the genome processing system can construct a genotype of the progeny based on the one or more informative reads and the one or more SNPs of interest in the first and second genetic sequence information. To do so, the genome processing system can scan through each of the identified informative reads in the progeny skim sequence and extract the corresponding haplotype blocks identified by the informative read identifier 145 from each parent. For example, if an informative read is identified as corresponding to the sire, the genome processing system can extract the haplotype block that includes the genome location of the informative read from the genome sequence of the sire (e.g., stored in the parent sequences). Likewise, if an informative read is identified as corresponding to the dam, the genome processing system can extract the haplotype block that includes the genome location of the informative read from the genome sequence of the dam (e.g., stored in the parent sequences). In some implementations, the genome processing system can utilize the FImpute algorithm to fill in additional gaps (e.g., regions of the progeny that are unsequenced) in the progeny skim sequence.

The genome processing system can perform imputation techniques, such as the FImpute algorithm (e.g., which may utilize many parent sequences from the species of the organism), following the extraction of the corresponding haplotype blocks from the parent sequences, which are then populated at corresponding positions in the generated progeny sequence. However, in some cases, gaps in the generated progeny sequence may remain, which may be populated using the FImpute algorithm and the parent sequences (or other genetic sequences of other members of the same species, which may be livestock). When implementing the FImpute algorithm, the genome processing system can utilize an overlapping sliding window to identify haplotype similarity between reference genetic sequences and the generated progeny sequence. In some implementations, the genome processing system can perform a phase cleaning technique over the generated progeny sequence. The generated progeny sequence is then stored in a memory, in association with an identifier of the embryo. It should be understood that while Fimpute is provided as an example, and that other imputation techniques may also be used to achieve desirable results.

Using the genotype information in the generated progeny sequence, the viability of the embryo can be determined. For example, the status of predetermined genes in the generated progeny sequence can identify genetic defects in the progeny, which if present may indicate that the progeny embryo is not viable. The genome processing system may scan through the generated progeny sequence to identify the status of such predetermined genes, and compare the statuses to a predetermined healthy baseline for each gene. If the genes match or are sufficiently similar to the predetermined healthy baseline, then the embryo can be identified as viable. If not, then the embryo can be identified as not viable. The genome processing system can calculate a score (e.g., a NetMerit index, etc.) for each embryo based on the genetic variation detected in the progeny sequence. This score may be calculated based on the variations of one or more SNPs corresponding to one or more traits of interest in the progeny. The score may be used to determine a course of action for the embryo, as described herein.

FIG. 4 shows the general architecture of an illustrative computer system 400 that may be employed to implement any of the computer systems discussed herein in accordance with some implementations. The computer system 400 can be used to provide information for display (e.g., genetic information related to one or more embryos). The computer system 400 of FIG. 4 comprises one or more processors 420 communicatively coupled to memory 425, one or more communications interfaces 405, and one or more output devices 410 (e.g., one or more display units) and one or more input devices 415. The processors 420 can be included in any of the computing device described herein.

In the computer system 400 of FIG. 4, the memory 425 may comprise any computer-readable storage media, and may store computer instructions such as processor-executable instructions for implementing the various functionalities described herein for respective systems, as well as any data relating thereto, generated thereby, or received via the communications interface(s) or input device(s) (if present). Referring again to the system 400 of FIG. 4, the computer system 400 can include the memory 425 to store information any of the information, variables, vectors, data structures, or other computer-readable information described herein, among others. The processor(s) 420 shown in FIG. 4 may be used to execute instructions stored in the memory 425 and, in so doing, also may read from or write to the memory various information processed and or generated pursuant to execution of the instructions.

The processor 420 of the computer system 400 shown in FIG. 4 also may be communicatively coupled to or control the communications interface(s) 405 to transmit or receive various information pursuant to execution of instructions. For example, the communications interface(s) 405 may be coupled to a wired or wireless network, bus, or other communication means and may therefore allow the computer system 400 to transmit information to or receive information from other devices (e.g., other computer systems). While not shown explicitly in the system of FIG. 4, one or more communications interfaces facilitate information flow between the components of the system 400. In some implementations, the communications interface(s) may be configured (e.g., via various hardware components or software components) to provide one or more user interfaces an access portal to at least some aspects of the computer system 400. Examples of communications interfaces 405 include user interfaces (e.g., web pages), through which the user can communicate with the computer system 400. In addition, the communication interface(s) 405 of the computing system 400 may communicate with other devices, such as sequencing devices or external storage systems.

The output devices 410 of the computer system 400 shown in FIG. 4 may be provided, for example, to allow various information to be viewed or otherwise perceived in connection with execution of the instructions. The input device(s) 415 may be provided, for example, to allow a user to make manual adjustments, make selections, enter data, or interact in any of a variety of manners with the processor during execution of the instructions. Additional information relating to a general computer system architecture that may be employed for various systems discussed herein is provided further herein.

Having now described useful embodiments and implementations for implementing the techniques described herein, some example applications of these techniques are now provided. It should be understood that the following examples may be implemented using any of the systems and methods described herein. Further, it should be understood these examples are purely for illustrative purposes, and should not be considered limiting to the scope of the systems, methods, or techniques described herein.

In an example, a non-invasive technique for the embryonic genotyping of embryos produced by IVF, or for the non-invasive genotyping of cells (such as cultured cells produced as part of a cell line) without requiring biopsy, cell culture, or cellular extractions may be used. In this example, methods and products of the invention may be used to amplify and genetically analyze cell-free DNA in the fluid IVF medium or in any other culture medium, and to make, for example, breeding selections and to breed animals based on those selections.

In this example, cell-free DNA may be obtained from any embryo or cell (e.g., from a cultured cell line) that has an outer membrane (or shell) permeable or selectively permeable membrane that DNA can traverse, wherein the embryo or cell requires growth, maturation, or culturing in a liquid medium. The liquid medium can either be a specialized growth medium or water. Any specialized growth medium known to be suitable for the embryos or cells in a particular application can be used. Serum free medium is advantageous as animal serum can contain DNA that may occasionally result in non-specific amplification products. For example, the animal serum may result in DNA contamination, making it difficult to differentiate the serum DNA from the embryo DNA, resulting in incorrect genotype calls. The present teachings are especially useful for non-human mammals and fish. Particularly livestock non-human mammals and fish.

The cell-free DNA obtained from the medium is then skim sequenced and phased using haplotype SNP-based genotyping as described herein above to impute the whole genome sequence of the embryo or cell from which the cell-free DNA was obtained.

It should be noted that certain passages of this disclosure may reference terms such as “first” and “second” in connection with devices, mode of operation, transmit chains, antennas, etc., for purposes of identifying or differentiating one from another or from others. These terms are not intended to merely relate entities (e.g., a first device and a second device) temporally or according to a sequence, although in some cases, these entities may include such a relationship. Nor do these terms limit the number of possible entities (e.g., devices) that may operate within a system or environment.

While the foregoing written description of the methods and systems enables one of ordinary skill to make and use what is considered presently to be the best mode thereof, those of ordinary skill will understand and appreciate the existence of variations, combinations, and equivalents of the specific embodiment, method, and examples herein. The present methods and systems should therefore not be limited by the above described embodiments, methods, and examples, but by all embodiments and methods within the scope and spirit of the disclosure.

Having now described some illustrative embodiments and implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed only in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation can be included in at least one implementation. Such terms as used herein are not necessarily all referring to the same implementation. Any implementation can be combined with any other implementation, inclusively or exclusively, in any manner consistent with the aspects and implementations disclosed herein.

References to “or” can be construed as inclusive so that any terms described using “or” can indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein can be embodied in other specific forms without departing from the characteristics thereof. Although the examples provided can be useful for embryo genotyping and using genomic data for genotype imputation, the systems and methods described herein can be applied to other environments. The foregoing implementations are illustrative rather than limiting of the described systems and methods. The scope of the systems and methods described herein can thus be indicated by the appended claims, rather than the foregoing description, and changes that come within the meaning and range of equivalency of the claims are embraced therein.

What is claimed is:

1. A method for computational genotype imputation, the method comprising:
maintaining, by one or more processors coupled to memory of a computing system, first genetic sequence information of a sire and second genetic sequence information for a dam, the first and second genetic sequence information indicating one or more single nucleotide polymorphisms (SNPs) of interest;
sequencing, by the one or more processors, based on a skim sequencing technique, a sample of genetic information of a progeny of the sire and the dam;
identifying, by the one or more processors, one or more informative variants based on the first genetic sequence information and the second genetic sequence information;
identifying, by the one or more processors, one or more informative reads in the sequence of the progeny based on the one or more informative variants; and
constructing, by the one or more processors, a genotype of the progeny based on the one or more informative reads and the one or more SNPs of interest in the first and second genetic sequence information.

2. The method of claim 1, wherein the progeny is an embryo and wherein the embryo is frozen based on the genotype of the embryo having one or more desirable genes or variants.

3. The method of claim 1, wherein the progeny is an embryo and wherein the embryo is terminated based on the genotype of the embryo having one or more undesirable genes or variants.

4. The method of claim 1, wherein the progeny is an embryo and wherein the embryo is implanted based on the genotype of the embryo having one or more desirable genes or variants.

5. The method of claim 1, wherein the progeny is an embryo and wherein the embryo is cloned based on the genotype of the embryo having one or more desirable genes or variants.

6. The method of claim 1, wherein the progeny is an embryo and wherein a cell line is created using the embryo based on the genotype of the embryo having one or more desirable genes or variants.

7. The method of claim 1, wherein the progeny is an embryo and wherein the embryo is split based on the genotype of the embryo having one or more desirable genes or variants.

8. The method of claim 1, wherein the progeny is an embryo and wherein the embryo is vitrified based on the genotype of the progeny.

9. The method of claim 1, wherein the progeny is an embryo and wherein the embryo is selected as a future sire or a future dam based on the progeny having one or more desirable genes or variants.

10. The method of claim 1, wherein sequencing the sample of the genetic information of the progeny further comprises sequencing, by the one or more processors, the sample of genetic information of the progeny at a low coverage corresponding to about 0.004×-2× coverage.

11. The method of claim 1, wherein identifying the one or more informative variants further comprises identifying a first phased region in the first genetic sequence information that is homozygous that corresponds to a second phased region in the second genetic sequence information that is heterozygous.

12. The method of claim 1, wherein identifying the one or more informative reads further comprises searching, by the one or more processors, the sequence of the progeny to identify matches with the one or more informative variants.

13. The method of claim 1, wherein constructing the genotype of the progeny further comprises constructing, by the one or more processors, the genotype of the progeny to include one or more haplotype blocks of the first genetic sequence information or the second genetic sequence information.

14. The method of claim 1, wherein the genotype of the progeny is constructed further based on a phased haplotype block.

15. The method of claim 1, wherein the sample consists of genetic information extracted from four or fewer cells.

16. The method of claim 1, further comprising performing a phase cleaning technique over the genotype of the progeny wherein the phase cleaning technique comprises correcting phase flipping by executing a median filter to correct incorrect phasing.

17. A system for computational genotype imputation after biopsy but prior to blastocyst hatching, the system comprising:
one or more processors coupled to a non-transitory memory, the one or more processors configured to:
maintain first genetic sequence information of a sire and second genetic sequence information for a dam, the first and second genetic sequence information indicating one or more single nucleotide polymorphisms (SNPs) of interest;
sequence, based on a skim sequencing technique, a sample of genetic information of a progeny of the sire and the dam;
identify one or more informative variants based on the first genetic sequence information and the second genetic sequence information;
identify one or more informative reads in the sequence of the progeny based on the one or more informative variants; and
construct a genotype of the progeny based on the one or more informative reads and the one or more SNPs of interest in the first and second genetic sequence information.

18. A non-transitory computer-readable storage medium having instructions embodied thereon, the instructions, when executed by one or more processors, cause the one or more processors to perform operations of constructing a genotype of a progeny embryo based on a sample of genetic information from the progeny embryo, the operations comprising:
maintaining first genetic sequence information of a sire and second genetic sequence information for a dam, the first and second genetic sequence information indicating one or more single nucleotide polymorphisms (SNPs) of interest;
sequencing, based on a skim sequencing technique, a sample of genetic information of a progeny embryo of the sire and the dam;
identifying one or more informative variants based on the first genetic sequence information and the second genetic sequence information;
identifying one or more informative reads in the sequence of the progeny embryo based on the one or more informative variants; and
constructing a genotype of the progeny after biopsy but prior to blastocyst hatching based on the one or more informative reads and the one or more SNPs of interest in the first and second genetic sequence information.
